(19) United States
(12) Patent Application Publication
(10) Pub. No.: US 2009/0324502 A1
(43) Pub. Date: Dec. 31, 2009

(54) Nasal Formulations of Insulin

(75) Inventors: Robert Stote, Gulfport, FL (US); Poul Strange, Princeton Junction, NJ (US)

Correspondence Address: AXINN, VELTROP & HARKRIDER LLP;Attn. Michael A. Davitz, 114 West 47th Street, New York, NY, 10036, US

(73) Assignee: CPEX Pharmaceuticals, Inc., Exeter, NH (US)

(21) Appl. No.: 12/479,348

(22) Filed: Jun. 5, 2009

Related U.S. Application Data
(60) Provisional application No. 61/059,225, filed on Jun. 5, 2008.

(51) Int. Cl. A61K 49/00; A61K 38/28; A61P 3/10

(52) U.S. Cl. 424/9.2; 514/3

(57) ABSTRACT

The present invention provides a method for achieving a therapeutically effective plasma levels of insulin by administering at least two doses of pharmaceutical formulation of insulin sequentially into the same nostril. The administration of the second dose in the same nostril gives substantially higher plasma levels of insulin when compared with sequential administration in two different nostrils. Without being limited to any specific physiological mechanism, it is believed that the first dose of insulin acts as a loading dose. This loading dose is required to achieve the subsequent plasma levels of insulin that are observed with subsequent doses. The Cmax of plasma insulin achieved by the methods and formulations of the present invention is at least about 70 microU/ml when plasma insulin is measured from about 0 to about 45 minutes after administration of a second dose. The AUC achieved is at least about 1800 microU/(ml*min). When administered sequentially in the same nostril, the Cmax of plasma insulin after a second dose is about five-fold greater than the Cmax of plasma insulin observed after the first dose; note, plasma insulin is measured from about 0 to about 45 minutes after administration of a second dose.

### CROSS REFERENCE TO RELATED APPLICATION

This application claims the benefit under 35 U.S.C. § 119(e) of the U.S. Provisional Application No. 61/059,225, filed on Jun. 5, 2008.

### FIELD OF THE INVENTION

The present invention relates to methods and formulations of insulin for nasal delivery.

### BACKGROUND OF THE INVENTION

Insulin is a hormone that induces transport of glucose from the blood to the inside of a cell, where the glucose provides a source of energy. People who suffer from Type I and Type 2 diabetes often require the administration of exogenous insulin to control blood sugar. Numerous studies have shown that tight regulation of blood glucose is critical to control the incidence and severity of many of the major complications of diabetes. Skyler. Clinical Diabetes 22(4):162-166 (2004). A key factor in maintaining blood glucose control, particularly in Type I diabetics, where there is limited or absent insulin production, is the timely delivery of insulin in doses that match the increase in blood glucose after a meal. If too much insulin is delivered or the timing of the delivered insulin does not adequately match the need, hypoglycemia can occur. In contrast, if too little insulin is delivered, hyperglycemia may result. Both conditions can cause serious clinical complications. The most common regimen of insulin treatment is subcutaneous injection of short term, fast acting insulin before meals in conjunction with administration of a longer, slower acting formulation of insulin. The end result of the combination of short and long acting insulins when closely monitored, is generally adequate; however, there is considerable variation among individuals in blood glucose control. In part, this variation is the result of variability in the release of insulin from the site of injection. Insulin uptake at site of injection is sensitive to skin temperature, vascularity and whether or not the underlying muscle is being exercised. Over time side effects of multiple injections, such as scarring and hypersensitivity of tissue at the injection site, can also lead to variability in insulin uptake from the site of injection.

Insulin may also be administered by inhalation. There are, however, disadvantages with this route of administration. Concerns regarding the potential for pulmonary toxicity with chronic use of inhaled insulin have been raised due to the growth-promoting and immunogenic properties of insulin. Moreover, reductions in lung function have been reported in both type 1 and type 2 diabetic patients. Mori et al, Internal Medicine, 31:189-93 (1992). The safety of inhaled insulin on pulmonary function has been the major concern throughout its clinical development.

Since the 1980s, there has been a great deal of interest in the potential of delivering insulin by nose. An advantage of this method of administration is that absorption of insulin by mucous membranes is direct, i.e., there is only a minimal barrier between the site of delivery and the circulation. Moreover, it has been found that certain agents which produce an antigenic effect when administered by injection do not do so when administered intra-nasally. Nasal spray dispensers are small in size and thus, more convenient than those used for pulmonary administration. Ease of use may lead to improved compliance, particularly in adolescent patients.

In general, the bioavailability of intra-nasally administered insulin has been poor (1-2%). Even with the addition of absorption enhancers to the formulation, absolute bioavailability has remained between 5 and 15%. Hinchcliffe et al, Drug Delivery Reviews. 35:199-234 (1999). In efforts to facilitate the development of intra-nasal formulations, a number of agents have been proposed as absorption enhancers. These have included bile salts and their derivatives, surfactants, fatty acids and their derivatives, and various bioadhesive molecules. Hinchcliffe et al, Drug Delivery Reviews. 35:199-234 (1999). However, poor bioavailability is still generally seen with intra-nasal delivery, which is further complicated by comparatively high inter-individual variability in the absorption of insulin via this route. Besides improving the nasal insulin formulation, there is a pressing need to optimize the insulin dosing technique and to improve the safety of insulin therapy by determining the ability of a patient to absorb insulin by nose.

### SUMMARY OF THE INVENTION

It is an object of the present invention to provide a method for achieving a therapeutically effective plasma level of insulin by administering at least about two doses of a pharmaceutical formulation of insulin sequentially into a single nostril. Each dose of the pharmaceutical formulation of insulin comprises at least about 10 international units (U) to about 100 U of insulin per 100 microliters. The dose may also comprise about 15 U to about 75 U, about 20 U to about 50 U, or about 25 U of insulin per 100 microliters. The plasma insulin is measured during the time period ranging from 0 to about 45 minutes, or from about 25 minutes to about 30 minutes after administration of the second dose. The maximum measured concentration of plasma insulin during the selected dosing interval (Cmax) after the second pharmaceutical dose is at least about 70 microU/ml. The area under the plasma concentration versus time curve (AUC) of plasma insulin after the second pharmaceutical dose is at least about 1800 microU/(ml*min). The Cmax (or AUC) of plasma insulin after the second dose is about two-fold to about ten-fold greater, about three-fold to about eight-fold greater, about four-fold to about five-fold greater, or about five-fold greater than the Cmax (or AUC) of plasma insulin after a first dose, when plasma insulin is measured during the time period ranging from 0 to about 45 minutes or from about 25 minutes to about 30 minutes after administration of the second dose. The Cmax (or AUC) of plasma insulin after a second dose is administered sequentially in the same nostril is about two-fold greater than the Cmax (or AUC) of plasma insulin observed where a second dose is administered sequentially in two different nostrils. The pharmaceutical formulation of insulin may comprise a therapeutically effective amount of insulin, a permeation enhancer, and a liquid carrier.

The present invention further provides a method for identifying a patient capable of absorbing a therapeutically effective amount of insulin comprising administering a dose of insulin by nose ranging from about 20 U to about 200 U in a pharmaceutical formulation and then measuring the plasma levels of insulin about 10 to about 30 minutes after administration of the dose. The doses may be broken down into multiple smaller doses, e.g., 4×25 U/dose. The dose of insulin may range from about 25 U to about 150 U, from about 50 U to about 125 U, or from about 75 U to about 110 U. The dose of insulin may also be about 100 U. A patient who absorbs a therapeutically effective amount of insulin has a Cmax of plasma insulin ranging from about 15 to about 400 microU/ml, from about 30 to about 250 microU/ml, from about 50 to about 150 microU/ml, from about 70 to about 100 microU/ml, or from about 15 to about 20 microU/ml. A preferred range for Cmax of a patient who absorbs a therapeutically effective amount of insulin is greater than about 70 microU/ml.

The present invention further provides an article of manufacture comprising a pharmaceutical formulation of insulin for nasal administration and printed matter indicating that, to achieve a therapeutically effective plasma level of insulin, at least about two doses of the pharmaceutical formulation of insulin should be administered sequentially in a single nostril. The printed matter states that the dose comprises at least about 10 U to about 100 U of insulin per 100 microliters. The dose may also comprise about 15 U to about 75 U, about 20 U to about 50 U, or about 25 U of insulin per 100 microliters. The pharmaceutical formulation of insulin may comprise a therapeutically effective amount of insulin, a permeation enhancer, and a liquid carrier. The printed matter states that the Cmax of insulin is at least about 70 microU/ml after a second dose is administered when plasma insulin is measured during the period from about 0 to about 45 minutes, or from about 25 minutes to about 30 minutes after administration. The printed matter also indicates that the AUC of plasma insulin after the second dose is at least about 1800 microU/(ml*min). The printed matter states that the Cmax (or AUC) of plasma insulin after a second dose is administered is about two-fold to about ten-fold greater, about three-fold to about eight-fold greater, about four-fold to about five-fold greater, or about five-fold greater than the Cmax (or AUC) of plasma insulin after a first dose has been administered.

The printed matter may also indicate that prior to administration of the insulin by formulation by nose, a patient should be evaluated to determine whether they are able to absorb a therapeutically effective amount of insulin by nose. The procedure comprises administering a dose of insulin by nose ranging from about 20 U to about 200 U in a pharmaceutical formulation and then measuring the plasma level of insulin about 10 to about 30 minutes after administration of the dose. The dose of insulin may also range from about 25 U to about 150 U, from about 50 U to about 125 U or from about 75 U to about 110 U. The dose of insulin may be about 100 U. A patient who absorbs a therapeutically effective amount of insulin has a Cmax of plasma insulin ranging from about 15 to about 400 microU/ml, from about 30 to about 250 microU/ml, from about 50 to about 150 microU/ml, from about 70 to about 100 microU/ml, or from about 15 to about 20 microU/ml. A preferred range for the Cmax of a patient who absorbs a therapeutically effective amount of insulin is greater than about 70 microU/ml. The printed matter states that the dosing of the pharmaceutical formulation of insulin is not dependent on how far an intra-nasal spray device is inserted into the nostril, whether the patient is inspiring or the angle of insertion of the intra-nasal spray.

The present invention further provides a method for identifying a patient who absorbs a therapeutically effective amount of insulin comprising administering a dose of insulin by nose ranging from about 20 U to about 200 U in a pharmaceutical formulation, providing a caloric challenge and then measuring the rise in plasma level of glucose about 15 to about 120 minutes after administration of the dose. The dose of insulin may also range from about 25 U to about 150 U, from about 50 U to about 125 U, or from about 75 U to about 110 U. The dose of insulin may be about 100 U. A patient with a rise in glucose of less than about 60 mg/dl within that time period would be considered to be a patient capable of absorbing a therapeutically effective amount of insulin by nose. The range of response to glucose rise may be less than about 60 mg/dl, less than about 40 mg/dl or less than about 20 mg/dl.

### BRIEF DESCRIPTION OF THE DRAWINGS

FIG. 1A depicts the dose-exposure relation of administering one, two or three doses of insulin sequentially in a single nostril. FIG. 1B depicts the systemic exposure ratios between the administrations of three different doses of insulin in the same nostril.

FIG. 2 depicts that higher systemic exposure is achieved after a second dose of insulin is administered in the same nostril as compared with administration sequentially in an opposite nostril to a first dose. FIGS. 2A and 2B depict Cmax and AUC of plasma insulin, respectively, after the plasma insulin levels are measured during the period ranging about 0 to about 45 minutes after the administration of a second dose.

FIG. 3 depicts the high variability among individuals in absorbing nasally administered insulin.

FIG. 4 depicts an euglycemic clamp study in which glucose metabolism occurs at peak insulin concentration of more than 70 microU/ml.

### DETAILED DESCRIPTION OF THE INVENTION

The present invention provides a method for achieving therapeutically effective plasma levels of insulin by administering at least two doses of a pharmaceutical formulation of insulin sequentially into the same nostril. The administration of the second dose in the same nostril gives substantially higher plasma levels of insulin when compared with sequential administration of a pharmaceutical dose in two different nostrils. Also encompassed by the methods and formulations of the present invention is a single administration of a dose of insulin in one nostril. Without being limited to any specific physiological mechanism, it is believed that the first dose of insulin acts as a loading dose for the nasal mucosa. This loading dose is required to achieve the plasma levels of insulin observed with subsequent doses. The Cmax of plasma insulin achieved by the methods and formulations of the present invention may be at least about 70 microU/ml when plasma insulin is measured during a period ranging from about 0 to about 45 minutes after administration of the second dose. The AUC achieved may be at least about 1800 microU/(ml*min). As used herein, the term “U” is equivalent to “IU”.

When administered sequentially in the same nostril, the Cmax of plasma insulin adjusted for baseline after a second dose is about five-fold greater than the Cmax of plasma insulin observed after the first dose; note, plasma insulin is measured from about 0 to about 45 minutes, or from about 25 minutes to about 30 minutes after administration of the second dose. When administered sequentially in the same nostril, the Cmax of plasma insulin after a third dose is about six-fold to about seven-fold greater than the Cmax of plasma insulin observed after the first dose, when plasma insulin is measured from about 0 to about 45 minutes, or from about 25 minutes to about 30 minutes after administration of the third dose. Similarly, the AUC of plasma insulin after a second dose is about five-fold greater than the AUC of plasma insulin observed after the first dose. The AUC of plasma insulin after a third dose is about six-fold to about seven-fold greater than the AUC of plasma insulin observed after the first dose. Depending on the dosage of insulin administered nasally, the Cmax (or AUC) of plasma insulin after the second dose may range from about two-fold to about ten-fold greater, about three-fold to about eight-fold greater, about four-fold to about five-fold greater, or about five-fold greater than the Cmax (or AUC) of plasma insulin observed after the first dose. The Cmax (or AUC) of plasma insulin after the third dose may range from about three-fold to about fifteen-fold greater, about four-fold to about twelve-fold greater, or about six-fold to about seven-fold greater than the Cmax (or AUC) of plasma insulin observed after the first dose.

The Cmax of plasma insulin for sequential administration in the same nostril is about two-fold greater than the Cmax of plasma insulin observed for sequential administration in two different nostrils (plasma insulin is measured during a similar time period, i.e., from about 0 to about 45 minutes, or from about 25 minutes to about 30 minutes after administration of the second dose of insulin). A similar difference is observed with the AUC after the second dose in the same nostril, where the AUC of plasma insulin after the second dose is administered sequentially in the single nostril is about two-fold greater than the AUC of plasma insulin observed where a second dose is administered sequentially in two different nostrils.

The pharmaceutical formulations of the present invention may comprise a pharmaceutically effective amount of insulin and a permeation enhancer. U.S. Pat. Nos. 7,112,561, 7,244,703 and 7,320,968. The permeation enhancer may be a Hsieh enhancer having the following structure:

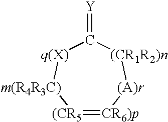

wherein X and Y are oxygen, sulfur or an imino group of the structure

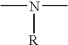

or ═N—R with the proviso that when Y is the imino group, X is an imimo group, and when Y is sulfur, X is sulfur or an imino group, A is a group having the structure

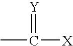

wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11, and said compound will enhance the rate of the passage of the drug across body membranes. Hereinafter these compounds are referred to as enhancers. When R, R₁, R₂, R₃, R₄, R₅ and R₆ is alkyl, it may be methyl, ethyl, propyl, isopropyl, butyl, isobutyl, sec-butyl, amyl, hexyl, and the like. Such permeation enhancers are described in U.S. Pat. No. 5,023,252 and U.S. Pat. No. 5,731,303.

Examples of the permeation enhancers are the cyclic lactones (the compounds wherein both X and Y are oxygen, (q is 1 and r is 0), the cyclic diesters (the compounds wherein both X and Y are oxygen, and both q and r are 1), and the cyclic ketones (the compounds wherein both q and r are 0 and Y is oxygen). In the cyclic diesters m+n is preferably at least 3. In the cyclic ketones m+n is preferably from 11 to 15 and p is preferably 0. Examples of the enhancers for use in the present invention are macrocyclic enhancers. The term “macrocyclic” is used herein to refer to cyclic compounds having at least 12 carbons in the ring, including: (A) macrocyclic ketones, for example, 3 methylcyclopentadecanone (muscone), 9-cycloheptadecen-1-one (civetone), cyclohexadecanone, and cyclopentadecanone (normuscone); and (B) macrocyclic esters, for example, pentadecalactones such as oxacyclohexadecan-2-one (cyclopentadecanolide, .omega.-pentadecalactone). Other permeation enhancers that may be used are the simple long chain esters that are Generally Recognized As Safe (GRAS) in the various pharmacopoeial compendia. These may include simple aliphatic, unsaturated or saturated (but preferably fully saturated) esters, which contain up to medium length chains. Non-limiting examples of such esters include isopropyl myristate, isopropyl palmitate, myristyl myristate, octyl palmitate, and the like. The enhancers are of a type that are suitable for use in a pharmaceutical composition. An artisan of ordinary skill will also appreciate that those materials that are incompatible with or irritating to mucous membranes should be avoided.

The enhancer is present in the composition at a concentration effective to enhance penetration of the insulin across the nasal mucosa. Various considerations should be taken into account in determining the amount of enhancer to be used. Such considerations include, for example, the amount of flux (rate of passage through the membrane) achieved and the stability and compatibility of the components in the formulations. The enhancer is generally used in an amount of about 0.01 to about 25% (w/w) the composition, more generally in an amount of about 0.1 to about 15% (w/w) the composition, and in some embodiments in an amount of about 0.5 to about 15% (w/w) the composition. U.S. Pat. No. 7,112,561.

The pharmaceutical formulations of the present invention may comprise a therapeutically effective amount of insulin, a permeation enhancer, and a liquid carrier. The present formulations may be at an acidic pH, such as no greater than a pH of 4.5. The liquid carrier is present in the composition in a concentration effective to serve as a suitable vehicle for the compositions of the present invention. In general, the carrier is used in an amount of about 40 to about 98% (w/w) of the composition and in some embodiments in an amount of about 50 to about 98% (w/w) of the composition.

Without being limited to any specific physiological mechanism, it is believed that the first dose of insulin acts as a loading dose for the nasal mucosa. The penetration enhancer in the first dose may transiently increase the permeability of the mucous layer or the membranes of the epithelial cells to insulin in the subsequent dose(s). The penetration enhancer in the first dose may also reversibly open the intercellular tight junctions of the epithelial cells so that more insulin is absorbed when additional doses are administered in the same nostril.

In one embodiment of the present invention, the nasal administration of insulin is through a nasal spray which uses water as the liquid carrier with insulin being dispersed or dissolved in the water in a therapeutically effective amount. In another embodiment, the permeation enhancer is emulsified in the aqueous phase that contains the insulin. The emulsification may be effected through the use of one or more suitable surfactants. Any suitable surfactant or mixture of surfactants can be used in the practice of the present invention, including, for example, anionic, cationic, and non-ionic surfactants. Examples of non-ionic surfactants are PEG-60 corn glycerides, PEG-20 sorbitan monostearate, phenoxy-poly(ethyleneoxy)ethanol, sorbitan monooleate, and the like. In general the surfactant is present in an amount less than about 2% (w/w) the composition. In another embodiment, the surfactant may be present in amounts less than about 1.5% (w/w), less than about 1.3% (w/w), less than about 1% (w/w), or less than about 0.3% (w/w).

Any type of insulin may be used with the methods and formulations of the present invention, including, without limitation, native insulin, i.e., purified from bovine or porcine sources, recombinant insulin, proinsulin, any insulin analogues, derivatives, polymorphs, metabolites, pro-drugs, salts, and/or hydrates. Examples of insulin analogues are human insulin, insulin lispro, insulin aspart, insulin glulisine, insulin glargine, and insulin detemir. Zinc salts of insulin may also be used. Derivatives of insulin include, insulin that has been modified at the internal or terminal amino acids, for example, lysine/proline-substituted insulin derivatives. Rapid, intermediate- and long-acting insulins may also be used with the methods and systems of the present invention. Bethel et al. Journal of the American Board of Family Practice. 18:199-204 (2005).

In general the insulin or insulin derivative is present in the pharmaceutical formulation in an amount ranging from about 0.01 to about 15% (w/w) of the composition. In one embodiment an amount of insulin ranging about 0.01 to about 10% (w/w) of the composition is used. Alternatively, the insulin is present in an amount of about 0.1 to about 5% (w/w) of the pharmaceutical formulation. The dose of insulin used may range from about 25 U to about 150 U, from about 50 U to about 125 U, from about 75 U to about 110 U or from about 25 U to about 50 U. In one dosing about 100 U of insulin is administered. The doses may be delivered in single or multiple doses. The doses may contain equal or different amounts of insulin. In one embodiment, 100 U of insulin is delivered in four equal doses of 25 U/dose.

The composition of the present invention is generally delivered through a nasal spray applicator. If intra-nasal application is desired, the composition may be placed in an intra-nasal spray-dosing device or atomizer and then, be applied by spraying it into the nostrils of a patient for delivery to the mucous membrane of the nostrils. A sufficient amount is applied to achieve the desired systemic or localized drug levels. Intranasal sprays deliver about 200 microliters, with 50-150 microliters being typically applied. In a preferred embodiment, there is about 100 microliters per dose. One or more nostrils may be dosed and the application may occur as often as desired or as often as is necessary. In the present invention, dosing occurs sequentially in a single nostril. In one embodiment, the nasal spray applicator is selected to provide droplets of the composition of a mean size from about 10 microns to about 200 microns. More generally, the droplet size is from about 30 microns to about 100 microns.

The insulin spray composition of the invention is generally employed in a dosing regimen that is dependent on the patient being treated. The frequency of use and the amount of the dose may vary from patient to patient. In general, dosing is in an amount from about 10 U to about 50 Upper dose with a total administration of about 100 U. In a preferred embodiment, there is about 15 U to 30 U per dose or per spray. The patient may receive multiple doses during the day. As known in the art, the treatment of a disease such as diabetes through insulin therapy varies from patient to patient. Based on known insulin therapy and the teachings herein, one skilled in the art such as a physician can select the dosing regimen and dosage for a particular patient or patients.

The patient is administered a single dose of insulin into a nostril. Dosing is independent of how far the intra-nasal spray device is inserted into the nostril, whether the patient is inspiring or the angle of insertion of the device. A dose may comprise at least about 10 U to about 100 U of insulin per 100 microliters. The dose may also comprise about 15 U to about 75 U, about 20 U to about 50 U, or about 25 U of insulin per 100 microliters. In one embodiment, 25 U of insulin in 100 microliters is administered into the nostril. After an appropriate time period when the amount of liquid has been absorbed, a second dose is administered sequentially into the same nostril. In one embodiment, the second dose may be administered within 1-5 seconds after administration of the first dose. The second dose may contain about 25 U; however, the amount of insulin in the first or second doses will be determined clinically and may vary.

After administration of nasal insulin, the plasma levels of insulin and glucose may be assayed to determine the maximum concentration of plasma insulin during the selected dosing interval (Cmax), the area under the plasma concentration versus time curve (AUC₀₋ₜ) and time to Cmax (Tmax). AUC may be measured from time 0 to time t, where a variety of time intervals may be selected. In one embodiment, blood is collected at time −5 minutes (5 minutes before administration of nasal insulin), −1 minute and 10, 15, 20, 25, 30 and 45 minutes after administration of nasal insulin. Insulin and C-peptides may be measured by immunoassay. Plasma glucose is measured using any standard laboratory chemistry means.

Pharmacokinetic (PK) parameters are derived from the relevant blood concentration data for glucose and insulin. Glucose and insulin are measured in samples taken from 0 to 45 minutes. The pharmacokinetic parameters for insulin include Cmax, AUC₀₋ₜ, Tmax, and comparison kinetics for all dosing to determine intra-subject variability and dose response. The pharmacokinetic parameters for glucose include AUC₀₋ₜ, comparison of consistency of dynamic effect under identical repeat dosing and with escalating dosing. The AUC may be calculated using a mixed log linear rule. Using this method the AUC is calculated by the trapezoid method between the first (data) point and Tmax and then by the logarithmic method between Tmax and the last data point. The calculation automatically switches to the trapezoidal method each time the concentration level increases or is equal between two data points. It is assumed that values below the limit of quantification (LOQ) which occur before Tmax are zero. Values below the limit of quantification, which occur after Tmax, are ignored for calculation of the terminal regression line. Interpolation between data points is allowed if a value below the limit of quantification, or a missing value, occurs between two values above the limit of quantification. The extrapolated area under the curve (t to infinity) is carried out using linear regression on the logarithmic (ln) ln-transformed data points of the curve with adjustment of t for estimation of the disposition rate constant (Lz). Each PK parameter is compared between treatments using an analysis of variance (ANOVA) model that includes the fixed effects of sequence, treatment and period, and the random effects of subjects within sequences and within-subject errors. Overall treatment effect is tested at 5% level of significance using the mean square error from the ANOVA model. If the overall treatment effect is not significant, then the between-treatment comparison will not be meaningful. The comparison between two treatments is made using the “ESTIMATE” statement of the SAS MIXED procedure. For each PK parameter, the descriptive statistics including N, mean, median, standard deviation, minimum and maximum are calculated by treatment group. For AUC and Cmax, in addition to the above-mentioned summary statistics, the geometric mean and coefficient of variation are also calculated by treatment group. Unless otherwise specified, all statistical tests are conducted against a two-sided alternative hypothesis, employing a significance level of 0.05.

There exists considerable inter-individual variability in the absorption of insulin via the intra-nasal route. Heinemann et al. Current Pharmaceutical Design. 7: 1327-1351 (2001). The present invention further provides a method for identifying a patient or group of patients who are capable of absorbing a therapeutically effective amount of insulin by nose. The insulin doses for such identification administered may range from about 20 U to about 200 U in a pharmaceutical formulation. After administration, the plasma levels of insulin are assayed about 10 to about 30 minutes after administration of the dose. The testing doses of insulin may range from about 25 U to about 150 U, about 50 U to about 125 U, about 75 U to about 110 U, or may be about 100 U. The Cmax of plasma insulin after administration may range from about 15 to about 400 microU/ml, from about 30 to about 250 microU/ml, from about 50 to about 150 microU/ml, from about 70 to about 100 microU/ml, from about 15 to about 20 microU/ml or may be greater than about 70 microU/ml (within an upper range of about 200 to 250 microU/ml). In specific embodiments of the present invention, the Cmax for a 100 U test dose is about 100 microU/ml, for a 75 U test dose about 67 microU/ml and for a 50 U dose about 30 microU/ml.

Alternatively, the blood glucose levels may be assayed after nasal administration of insulin. Blood glucose may be determined using standard methodology (Blood Sugar [online], [retrieved on Jun. 5, 2009]. Retrieved from the Internet <URL: http://en.wikipedia.org/wiki/Blood_sugar>). Additionally, identification of patients who absorb a therapeutically effective amount of insulin by nose may be performed with or without any caloric challenge. For example, patients in the fasting state would be tested for their baseline plasma glucose. Typically, that value would range from about 100-250 mg/dl. After appropriate training with nasal sprays, the patients would then receive a dose of insulin by nose. The insulin doses administered may range from about 20 U to about 200 U in a nasally compatible pharmaceutical formulation. After administration, the plasma levels of insulin are assayed about 10 to about 30 minutes after administration of the dose. The dose of insulin may range from about 25 U to about 150 U, from about 50 U to about 125 U, from about 75 U to about 110 U, or may be about 100 U.

Plasma glucose would then be measured from about 15 to about 120 minutes after administration of the insulin by nose. If the plasma glucose is less than the baseline glucose, then the patient is considered able to absorb a therapeutically effective amount of insulin by nose or may be sufficiently sensitive to insulin to result in decrease in plasma glucose Alternatively, the assays may be conducted using a standard caloric challenge, e.g., with a solid or liquid carbohydrate containing food or drink. For example, a 75 gm glucose containing drink could be used. Patients in a fasting state would have a baseline test of plasma glucose done. As noted, plasma glucose could range from about 100 to about 250 mg/dl. The dose ranges of insulin are described above. Immediately after receiving the nasal insulin, the patients would get the caloric challenge. Plasma glucose would be measured again during a time period ranging from about 10 to about 120 minutes after the caloric challenge. A patient with a rise in glucose of less than about 60 mg/dl would be considered to be a patient capable of absorbing a therapeutically effective amount of insulin by nose. The range of response to glucose rise may be less than about 60 mg/dl, less than about 40 mg/dl or less than about 20 mg/dl.

The present invention further provides an article of manufacture such as a kit comprising a pharmaceutical formulation of insulin for nasal administration and printed matter indicating that to achieve a therapeutically effective plasma level of insulin at least about two doses of the pharmaceutical formulation of insulin should be administered sequentially in a single nostril. The printed matter states that a dose comprises at least about 10 U to about 100 U of insulin per 100 microliters. The dose may also comprise about 15 U to about 75 U, about 20 U to about 50 U, or about 25 U of insulin per 100 microliters. The printed matter states that Cmax of insulin may range from about 15 to about 400 microU/ml, from about 30 to about 250 microU/ml, from about 50 to about 150 microU/ml, from about 70 to about 100 microU/ml, from about 15 to about 20 microU/ml or may be greater than about 70 microU/ml after a second dose is administered when plasma insulin is measured from about 0 to about 45 minutes, or from about 25 minutes to about 30 minutes after administration of the second dose of insulin and that the AUC of plasma insulin after the second dose is at least about 1800 microU/(ml*min). The printed matter also indicates that Cmax (or AUC) of plasma insulin after a second dose is administered is about two-fold to about ten-fold greater, about three-fold to about eight-fold greater, about four-fold to about five-fold greater, or about five-fold greater than Cmax (or AUC) of plasma insulin after a first dose is administered.

The printed matter may also indicate that patients should be tested to identify a patient or group of patients who absorb a therapeutically effective amount of insulin by nose. The dose of insulin administered by nose may range from about 20 U to about 200 U in a pharmaceutical formulation acceptable for nasal administration. The plasma insulin may be measured from about 10 to about 30 minutes after administration of the dose. The dose of insulin may range from about 25 U to about 150 U, from about 50 U to about 125 U, from about 75 U to about 110 U, or may be about 100 U. After administration of the nasal insulin, the Cmax of plasma insulin ranges from about 15 to about 400 microU/ml, from about 30 to about 250 microU/ml, from about 50 to about 150 microU/ml, from about 70 to about 100 microU/ml, from about 15 to about 20 microU/ml or may be greater than about 70 microU/ml (within an upper range of about 200 to 250 microU/ml).

Alternatively, the printed matter may indicate that plasma glucose should be assayed after administration of nasal insulin to determine whether the patient is able to absorb a therapeutically effective amount of insulin by nose. The printed matter may indicate that assay should be conducted using a standard caloric challenge, e.g., with solid or liquid carbohydrate containing food or drink. For example, a 75 gm glucose containing drink could be used. Patients in a fasting state would have a baseline test of plasma glucose tested. Immediately after receiving the nasal insulin, the patients would get the caloric challenge. Plasma glucose would be measured again from about 10 to about 120 minutes after the caloric challenge. A patient with a rise in glucose of less than about 60 mg/dl would be considered a patient capable of absorbing a therapeutically effective amount of insulin by nose. The range of response to glucose rise may be less than about 60 mg/dl, less than about 40 mg/dl or less than about 20 mg/dl.

As known in the art, the treatment of a disease such as diabetes through insulin therapy is dependent on the patient being treated. The printed matter incorporated into the article of manufacture may indicate that dosing of the pharmaceutical formulation of insulin is independent of how far an intra-nasal spray device is inserted into the nostril, whether the patient is inspiring or the angle of insertion of the intra-nasal spray.

Any pharmaceutically active agent, or a mixture of two or more such agents, capable of being delivered across a mucous membrane may be used in the practice of the present invention. The term “pharmaceutically active agent” includes peptides, proteins, peptidomimetics, peptoids, and chemical compounds, as well as precursors, salts, complexes, analogues, and derivatives of said peptides, proteins, peptidomimetics, peptoids, and chemical compounds. The agent may be therapeutic, prophylactic, or diagnostic in nature.

Examples of pharmaceutically active agents which may be employed in the practice of the present invention include: compounds useful in the treatment of diabetes, for example, insulin, proinsulin, preproinsulin, insulin analogues and glucagon-like peptides (GLPs); calcitonin and calcitonin gene-related peptides; growth hormones; growth hormone-releasing agents; cancer-treating agents, for example, somatostatins (SRIFs) and analogs thereof; gonadotropin-releasing agents (GnRHs—also known as luteinizing hormone-releasing hormone agonists (LHRHs)); gonadotropin-releasing hormone antagonists, for example, Antide; delta-sleep-inducing peptides (DSIPs); opioids; anti-obesity agents; anti-inflammatory agents; angiogenin antagonists; anti-opiate peptides, for example, morphine-modulating neuropeptides; beta-antagonists, for example, albuterol; anxiolytic agents, for example, diazepam, midazolam, barbiturates, paroxetine, imipramine, and related psychotrophic compounds; beta-blockers; appetite-enhancing compounds; narcotic and opioid analgesics; sex hormones, for example, testosterone, progesterone, and estradiol; and metabolic regulating peptides, for example, parathyroid hormone (PTH), thyroid stimulating hormone, thymic humoral factor (THF), and follicle stimulating hormone (FSH). WO 03/000158.

In embodiments of the present invention which comprise a peptide or a protein, the composition may comprise also an enzyme inhibitor which is capable of preventing the breakdown of a peptide or protein, for example, at the site of absorption. Essentially any suitable enzyme inhibitor or mixture of enzyme inhibitors may be used in the practice of the present invention. Example of enzyme inhibitors which may be used in the practice of the present invention are leupeptin, bestatin, and aprotinin. Depending on the enzymatic cleavage site in any given peptide or protein, different enzyme inhibitors may be used. The enzyme inhibitor can be used in a concentration effective to inhibit enzymatic degradation at the site of administration. For guideline purposes, it is believed most applications will involve the use of the enzyme inhibitor in an amount of about 0.0001 to about 1.0% (w/w) of the composition and more likely about 0.005 to about 0.1% (w/w) of the composition. WO 03/000158. The Examples illustrate embodiments of the invention and are not to be regarded as limiting.

### Example 1

This study's objective was to determine the optimal methodology for nasal administration of insulin, and to characterize dose response pharmacokinetics and pharmacodynamics. This study was performed following a protocol approved by the Institutional Review Board (IRB).

### Formulation and Device

The formulation tested was intra-nasal insulin spray containing regular short acting human recombinant insulin dissolved in water in combination with several common excipients, including Polysorbate 20, sorbitan monolaurate, cottonseed oil, and cylopentadecalactone (CPE-215). The excipient cyclopentadecalactone is a compound that occurs naturally in plants, such as Angelica archangelica, and is a common constituent of many foodstuffs, cosmetics and personal hygiene products. Importantly, the insulin formulation was brought to room temperature 2 to 10 hours before use. It was gently inverted two to three times. The pump was primed the very first time the spray was used. Each 100 microliter spray dispensed approximately 25 U of insulin. The Advanced Preservative Free (APF) nasal spray device kept the dose volume in the tolerance range for a period of at least one week.

### Study Subjects

Eight healthy, non-smoking male subjects between 18 and 50 years of age, and fitting the inclusion/exclusion criteria, participated in the study. Subjects had to meet all the following inclusion criteria: current history and physical (including nasal cavity exam) without clinically significant abnormalities; body mass index of ≦33; weight ≧70 kg; C-peptide level >1.0 mg/ml; agreement to the written informed consent prior to admission to the study.

Subjects were excluded if they met any one of the following exclusion criteria: presence of significant cardiac, gastrointestinal, endocrine, neurological, liver or kidney disease; history of present conditions known to interfere with the absorption, distribution, metabolism, or excretion of insulin; chronic use of medications for any reason (stable vitamin/nutritional supplements were allowed); had a fasting plasma glucose ≧126 mg/dl; had elevated liver enzymes (ALT, AST, alkaline phosphatase) >1.5 times the upper normal limit; had a history of drug or alcohol abuse within the last 2 years; use of investigational drug within 30 days prior to first treatment visit; routine use of a nasal spray; urinalysis positive for drugs of abuse at screening visit.

### Study Procedures

Subjects were treated according to the schedule tabulated in Table 1. Vital signs were obtained after the subject had been sitting for at least 5 minutes. Blood pressure and heart rate were obtained and recorded prior to each dose administration and as needed during the treatment day. A standardized meal consisting of approximately 600 kcals (50% CHO, 30% Fat, 20% Protein) were provided 45 minutes after each treatment with insulin and after the last blood draw.

TABLE 1

| Day 1 | Day 2 | Day 3 | Day 4 | Day 5 |
|---|---|---|---|---|
| Randomly dose 4 | Analyze | Randomly dose 4 | Analyze | Repeat |
| times over the day | data | times over the day | data | previous |
| with 25, 50, 75 & | | with 25, 50, 75 & | | dosing day |
| 100 U using the | | 100 U using both | | |
| same nostril | | nostrils | | |

During the study, all subjects fasted overnight for at least 8 hours prior to the morning treatments. During the overnight fast, water was allowed to be taken ad libitum, up to one hour pre-dosing. Subjects received their intra-nasal doses or self administered while sitting upright.

Subjects were administered according to the following protocol. Subjects determined whether both nostrils were open by alternatively pressing the other nostril and breathing in. If both nostrils were open, subjects determined if one was more open than the other. If so, then that was the nostril that was to be dosed with the 25 U (one spray) dose. If both nostrils were totally blocked, the subject gently blew his nostrils until at least one was clear. When administering the 50, 75, and 100 U doses (two or more sprays), both nostrils were ensured to be open (by blowing the nose gently if needed) and there were approximately 10-20 seconds between sprays. The subjects were dosed in a sitting position with the head bent forward toward the chest. This ensured proper dip tubefill for each dose. After the pump sprayer had been primed, the actuator was placed into the nostril.

Subjects closed the nostril that was not currently being dosed. For each spray, subjects or a medical care worker pressed firmly downward on the shoulders of the white applicator using forefinger and middle finger while supporting the base with thumb.

Simultaneously, as the spray was administered, the subject breathed gently inward or took a hard sniff through the nostril. The subject then breathed out through the mouth. The inward and outward breath was repeated as necessary. Each subject practiced the sniffing technique before receiving their doses. Subjects were not allowed to blow their noses for 30 minutes after dosing. Note, any of the above instructions may be incorporated into printed matter which can be handed to the patient as part of a kit.

### Sample Collection and Assay Methods

A 4 ml venous blood sample was aseptically aspirated (via an intravenous cannula) from each patient volunteer according to the schedule outlined below: Pharmacokinetics (PK) insulin (8 samples): −5, −1, 10, 15, 20, 25, 30, 45 minutes; PK glucose (8 samples): −5, −1, 10, 15, 20, 25, 30, 45 minutes; Bedside glucose determination during treatment (4 samples): *−5, 30, 40, 45 minutes. (* −5 minute glucose reading must be less than 126 mg/dl to proceed with dosing.)

Blood samples were collected into a fluoride oxalate tube for the determination of plasma glucose. These were bunched and run in the same analytical batch. Another sample was collected into plain tubes (no anticoagulant) and allowed to clot on the bench at room temperature for 30 minutes. The serum was separated and divided into duplicate, labeled polypropylene tubes which were frozen in dry ice and stored upright at −70° C. Blood cells after centrifugation were discarded. All insulin samples were bunched for each study day and run in the same analytical batch.

The concentrations of insulin and C-peptide in each blood sample were measured by immunoassay. Insulin concentrations were measured in samples taken from 0 to 45 minutes only and C-peptide for screening purposes only. The validation procedure followed international guidelines.

### Results for Insulin

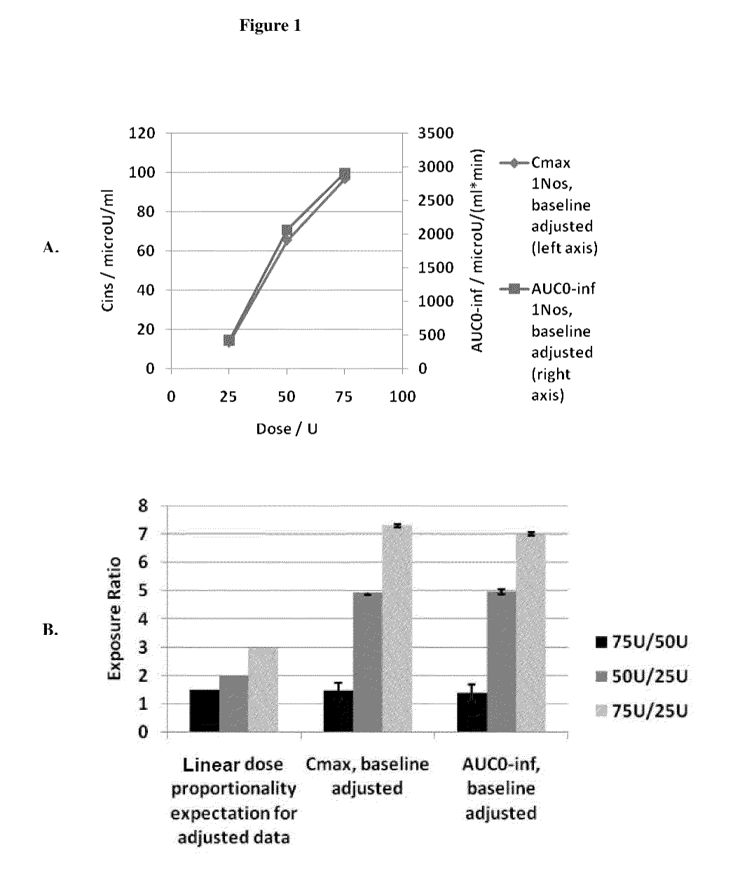

In FIG. 1A, Cmax or AUC of plasma insulin is plotted against insulin doses to show the dose-exposure relation after a single administration of insulin at each of three different doses (25, 50, and 75 U) in the same nostril. Exposure is the amount of insulin that is absorbed into the system circulation. The administration of the second 25 U in the same nostril (total dose was 50 U) achieved more than double the Cmax or AUC as compared with only a single dose of 25 U. In fact, typically about five times higher exposure was achieved after exposure of a single dose in the same nostril. Additive exposure of the third 25 U administration (total dose was 75 U) was roughly proportional, resulting in 48% and 41% increment of Cmax and AUC, respectively, over the 50 U administration. FIG. 1B shows the ratio of the incremental exposures as measured by Cmax and AUC when each of three different doses (25, 50, and 75 U) were administered in the same nostril. On the left hand of the graph, the expected change in the exposure is shown. Thus, for example, one would expect a two-fold increase in either AUC or Cmax or linear proportionality, if two doses, i.e., 50 U (2×25 U), were administered in the same nostril. However, when two sprays of 25 U each (total dose was 50 U) were administered in the same nostril, there was about a five-fold increase in Cmax or AUC when compared to a single spray of 25 U.

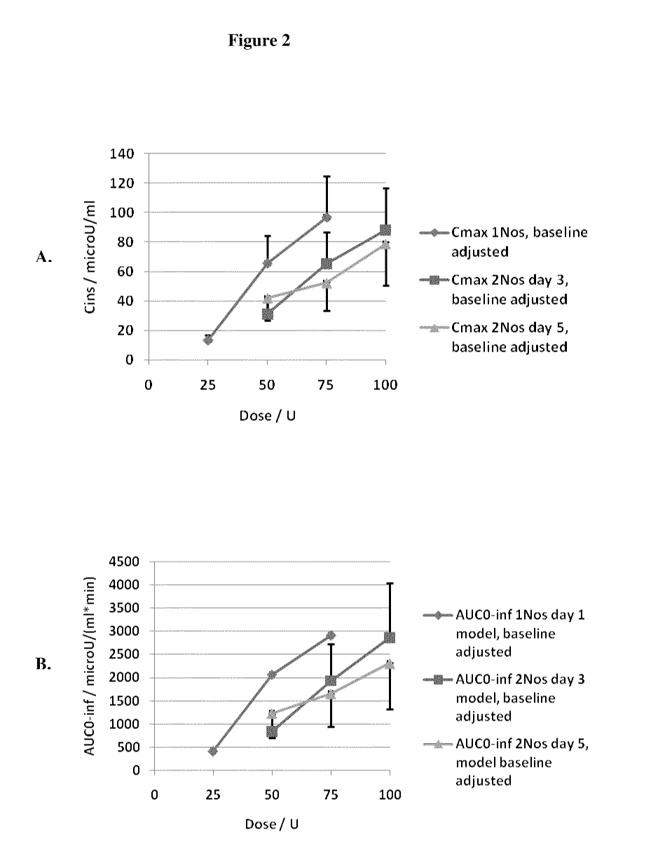

FIGS. 2A and 2B show the dose-exposure relation after two repeats of administration of three different doses (50, 75, and 100 U) in two nostrils (Cmax 2nos) or a single administration of three different doses (25, 50, and 75 U) in the same nostril (Cmax 1nos). Administration of the second 25 U dose in the same nostril achieved about a five-fold greater exposure as compared to the first dose, reflected by the Cmax in FIG. 2A and AUC in FIG. 2B, while the second 25 U dose when administered sequentially in two different nostrils only achieved about double the exposure.

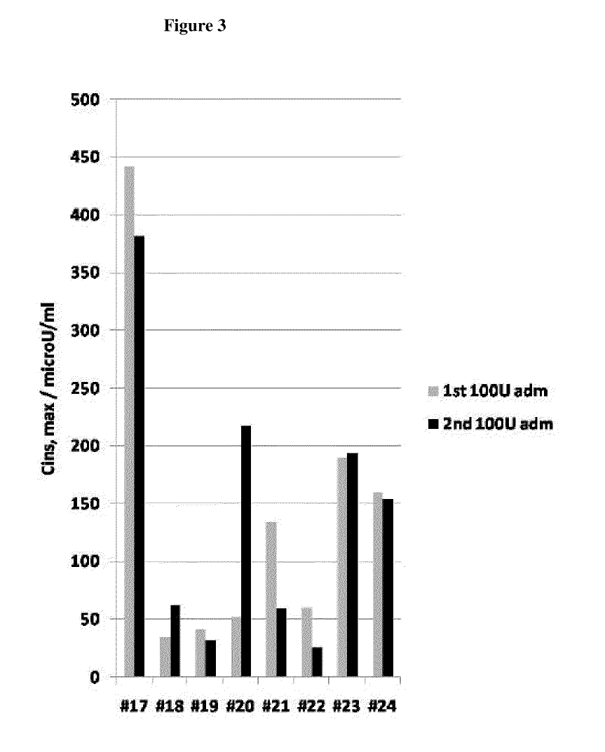

FIG. 3 shows the high variability between individuals in absorbing nasally administered insulin, which can be reflected as shown by a 15 fold difference in Cmax of plasma insulin among individuals. #17 to #24 refer to different study subjects. The intra-subject variability between two repeats of the same dosing schedule appears lower. This inter-subject variability had been previously attributed to administration technique, such as how far an intra-nasal spray device was inserted into the nostril, whether the patient was inspiring or angle of insertion of the intra-nasal spray. However, this study refuted those possibilities, and demonstrated that the inter-individual variability was more likely due to the existence of patients who are capable of absorbing therapeutically effective amounts of insulin across the nasal mucosa as opposed to those patients who absorb significantly less insulin by this means of administration. Therefore, to improve the safety of insulin therapy, it is crucial that, prior to insulin administration, a patient be assayed to determine the absorption of insulin after nasal administration.

### Example 2

Glucose clamp technique is a well established measure of immediate insulin action on glucose uptake. This measurement is achieved by clamping or maintaining a predetermined blood glucose level (e.g. ˜100 mg/dl) and is accomplished by using an adjusted glucose infusion rate to counter the action of the insulin being tested. The glucose infusion rate (GIR) therefore becomes a direct measure of the amount of glucose that “disappears” from plasma per unit of time.

The glucose clamp study was done according to the following procedure:

a. The subject fasted (except water) since 11:00 PM the previous night.
b. The vital signs were taken after subject rested for 5 min in sitting position.
c. Both arms were placed in heating pads for vein dilatation. In one arm an IV catheter was placed into the antecubital vein for infusion of Dextrose 20% and insulin via two separate stopcocks. The other IV catheter was placed retrograde for sampling of arterialized blood for glucose measurements. Heating pad could be removed from the glucose infusion site, but retrograde catheter site was maintained at 65° C. An initial blood (−30 min) was obtained to sample for baseline glucose. 30 min later insulin infusion was started to help ensure proper flow for clamp procedure.
d. 1 hour prior to dosing, normal saline was infused at a rate of 30 cc/hr.
e. Samples were obtained for blood glucose every 5 min throughout the clamp procedure. Stat was analyzed using the YSI 2300 Glucose Analyzer. Glucose infusion rate was adjusted as needed to maintain blood glucose at a constant level of 90-110 mg/dl.
f. Blood samples were obtained for glucose and insulin levels at −10, −1, 3, 6, 9, 12, 15, 20, 25, 30, 35, 40, 45, 50, 55, 60, 65, 70, 75, 80, 90, 120, 150, 180 and 240 min. C-peptide was sampled at −10, −1, 60, 120 and 240 minutes (5 total time points) for each dose.
g. Dextrose 20% infusion was continued for an additional 15 min.
h. Vital signs were obtained.
i. Meal was given to the subject and subject's blood glucose level was ensured to be greater than 100 mg/dl before discharge.

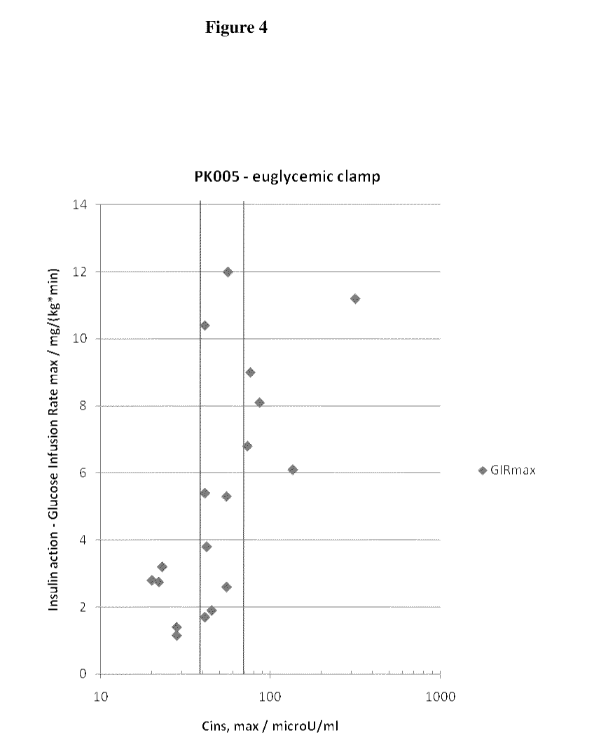

FIG. 4 plots the maximal achieved GIR as a function of Cmax measured. The data show that substantial glucose metabolism occurs at a Cmax of insulin greater than about 70 microU/ml.

The scope of the present invention is not limited by what has been specifically shown and described hereinabove. Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety. Variations, modifications and other implementations of what is described herein will occur to those of ordinary skill in the art without departing from the spirit and scope of the invention. While certain embodiments of the present invention have been shown and described, it will be obvious to those skilled in the art that changes and modifications may be made without departing from the spirit and scope of the invention. The matter set forth in the foregoing description and accompanying drawings is offered by way of illustration only and not as a limitation.

What is claimed is:

1. A method of achieving a therapeutically effective plasma level of insulin comprising administering at least about two doses of a pharmaceutical formulation of insulin sequentially into a single nostril.

2. The method of claim 1 wherein a dose comprises at least about 10 international units (U) to about 100 U of insulin per 100 microliters.

3. The method of claim 2 wherein Cmax of plasma insulin is at least about 70 microU/ml when plasma insulin is measured from about 0 to about 45 minutes after administration of a second dose.

4. The method of claim 3 wherein AUC of plasma insulin after the second dose is at least about 1800 microU/(ml*min).

5. The method of claims 1 or 2 wherein Cmax of plasma insulin after a second dose is about two-fold to about ten-fold greater than Cmax of plasma insulin after a first dose when plasma insulin is measured from about 0 to about 45 minutes after administration of a second dose.

6. The method of claim 5 wherein Cmax of plasma insulin after a second dose is about three-fold to about eight-fold greater than Cmax of plasma insulin after a first dose.

7. The method of claim 6 wherein Cmax of plasma insulin after a second dose is about five-fold greater than Cmax of plasma insulin after a first dose.

8. The method of claim 5 wherein AUC of plasma insulin after a second dose is about two-fold to about ten-fold greater than AUC of plasma insulin after a first dose.

9. The method of claim 8 wherein AUC of plasma insulin after a second dose is about three-fold to about eight-fold greater than AUC of plasma insulin after a first dose.

10. The method of claim 9 wherein AUC of plasma insulin after a second dose is about five-fold greater than AUC of plasma insulin after a first dose.

11. The method of claim 1 wherein Cmax of plasma insulin after a second dose is administered sequentially in the same nostril is at least about two-fold greater than Cmax of plasma insulin observed where a second dose is administered sequentially in two different nostrils, wherein plasma insulin is measured from about 0 to about 45 minutes after administration of the second dose of insulin.

12. The method of claim 11 wherein AUC of plasma insulin after the second dose is administered sequentially in the same nostril is at least about two-fold greater than AUC of plasma insulin observed where a second dose is administered sequentially in two different nostrils.

13. The method of claim 1 wherein the pharmaceutical formulation of insulin comprises: a therapeutically effective amount of insulin, a permeation enhancer, and a liquid carrier, said permeation enhancer being a Hsieh enhancer having the following structure: 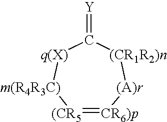
wherein X and Y are oxygen, sulfur or an imino group of the structure 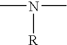
or ═N—R with the proviso that when Y is the imino group, X is an imimo group, and when Y is sulfur, X is sulfur or an imino group, A is a group having the structure 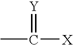
wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11.

14. A method of identifying a patient capable of absorbing a therapeutically effective amount of insulin comprising administering a dose of insulin by nose ranging from about 20 U to about 200 U in a pharmaceutical formulation and then measuring plasma level of insulin about 10 to about 30 minutes after administration of the dose.

15. The method of claim 14 wherein the dose of insulin ranges from about 25 U to about 150 U.

16. The method of claim 15 wherein the dose of insulin ranges from about 50 to about 125 U.

17. The method of claim 16 wherein the dose of insulin ranges from about 75 U to about 110 U.

18. The method of claim 17 wherein the dose of insulin is about 100 U.

19. The method of claim 14 wherein Cmax of plasma insulin ranges from about 15 to about 400 microU/ml.

20. The method of claim 19 wherein the Cmax of plasma insulin ranges from about 30 to about 250 microU/ml.

21. The method of claim 20 wherein the Cmax of plasma insulin ranges from about 50 to about 150 microU/ml.

22. The method of claim 19 wherein the Cmax of plasma insulin is greater than about 70 microU/ml.

23. An article of manufacture comprising a pharmaceutical formulation of insulin for nasal administration and printed matter indicating that, to achieve a therapeutically effective plasma level of insulin, at least about two doses of the pharmaceutical formulation of insulin should be administered sequentially in a single nostril.

24. The article of manufacture of claim 23 wherein the pharmaceutical formulation of insulin comprises: a therapeutically effective amount of insulin, a permeation enhancer, and a liquid carrier, said permeation enhancer being a Hsieh enhancer having the following structure: 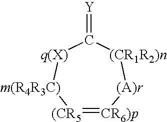
wherein X and Y are oxygen, sulfur or an imino group of the structure 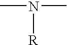
or ═N—R with the proviso that when Y is the imino group, X is an imimo group, and when Y is sulfur, X is sulfur or an imino group, A is a group having the structure 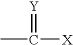
wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11.

25. The article of manufacture of claim 23 wherein the printed matter states that a dose comprises at least about 10 U to about 100 U of insulin per 100 microliters.

26. The article of manufacture of claim 25 wherein the dose comprises about 15 U to about 75 U of insulin per 100 microliters.

27. The article of manufacture of claims 25 or 26 wherein the dose comprises about 25 U of insulin per 100 microliters.

28. The article of manufacture of claim 23 wherein the printed matter states that Cmax of insulin is at least about 70 microU/ml after a second dose is administered when plasma insulin is measured from about 0 to about 45 minutes.

29. The article of manufacture of claim 28 wherein the printed matter states that AUC of plasma insulin after the second dose is at least about 1800 microU/(ml*min).

30. The article of manufacture of claim 23 wherein the printed matter states that Cmax of plasma insulin after a second dose is administered is about two-fold to about ten-fold greater than Cmax of plasma insulin after a first dose is administered.

31. The article of manufacture of claim 30 wherein the printed matter states that AUC of insulin after the second dose is about two-fold to about ten-fold greater than AUC of insulin after the first dose.

32. An article of manufacture comprising a pharmaceutical formulation of insulin for nasal administration and printed matter indicating that prior to administration of the pharmaceutical formulation, a patient should be evaluated to determine whether the patient is able to absorb a therapeutically effective amount of insulin comprising administering a dose of insulin by nose ranging from about 20 U to about 200 U in a pharmaceutical formulation and then measuring plasma level of insulin about 10 to about 30 minutes after administration of the dose.

33. The article of manufacture of claim 32 wherein the dose of insulin ranges from about 25 U to about 150 U.

34. The article of manufacture of claim 33 wherein the dose of insulin ranges from about 50 U to about 125 U.

35. The article of manufacture of claim 34 wherein the dose of insulin ranges from about 75 U to about 110 U.

36. The article of manufacture of claim 35 wherein the dose of insulin is about 100 U.

37. The article of manufacture of claim 32 wherein Cmax of insulin ranges from about 15 to about 400 microU/ml.

38. The article of manufacture of claim 37 wherein the Cmax of insulin ranges from about 30 to about 250 microU/ml.

39. The article of manufacture of claim 38 wherein the Cmax of insulin ranges from 50 to about 150 microU/ml.

40. The article of manufacture of claim 32 wherein the Cmax is greater than about 70 microU/ml.

41. The article of manufacture of claims 32 or 40 wherein the pharmaceutical formulation of insulin comprises: a therapeutically effective amount of insulin, a permeation enhancer, and a liquid carrier, said permeation enhancer being a Hsieh enhancer having the following structure: 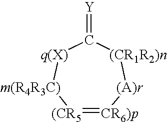
wherein X and Y are oxygen, sulfur or an imino group of the structure 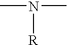
or ═N—R with the proviso that when Y is the imino group, X is an imimo group, and when Y is sulfur, X is sulfur or an imino group, A is a group having the structure 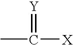
wherein X and Y are defined above, m and n are integers having a value from 1 to 20 and the sum of m+n is not greater than 25, p is an integer having a value of 0 or 1, q is an integer having a value of 0 or 1, r is an integer having a value of 0 or 1, and each of R, R₁, R₂, R₃, R₄, R₅ and R₆ is independently hydrogen or an alkyl group having from 1 to 6 carbon atoms which may be straight chained or branched provided that only one of R₁ to R₆ can be an alkyl group, with the proviso that when p, q and r have a value of 0 and Y is oxygen, m+n is at least 11, and with the further proviso that when X is an imino group, q is equal to 1, Y is oxygen, and p and r are 0, then m+n is at least 11.

42. The article of manufacture of claim 32 wherein the printed material further states that dosing of the pharmaceutical formulation is not dependent on how far an intra-nasal spray device is inserted into the nostril, whether the patient is inspiring or angle of insertion of the intra-nasal spray.

43. A method of identifying a patient capable of absorbing a therapeutically effective amount of insulin comprising administering a dose of insulin by nose ranging from about 20 U to about 200 U in a pharmaceutical formulation, providing a caloric challenge and then measuring the rise in plasma level of glucose about 15 to about 120 minutes after administration of the dose.

44. The method of claim 43 wherein the dose of insulin ranges from about 25 U to about 150 U.

45. The method of claim 44 wherein the dose of insulin ranges from about 50 U to about 125 U.

46. The method of claim 45 wherein the dose of insulin ranges from about 75 U to about 110 U.

47. The method of claim 46 wherein the dose of insulin is about 100 U.

48. The method of claim 43 wherein rise in plasma glucose is less than about 60 mg/dl.

49. The method of claim 48 wherein the rise is less than about 40 mg/dl.

50. The method of claim 49 wherein the rise is less than about 20 mg/dl.
